(19) United States
(12) Patent Application Publication
(10) Pub. No.: US 2023/0270900 A1
(43) Pub. Date: Aug. 31, 2023

(54) CLEANING KIT HAVING A SOLUTE

(71) Applicant: Berkshire Holding Corporation, Whitsett, NC (US)

(72) Inventors: Wenyu Zhang, Durham, NC (US); Susan E. Faile, Asheboro, NC (US); Justin J. Autry, Durham, NC (US); Whitmore B. Kelley, Lebanon, NH (US); David Capotosto, Biddeford, ME (US); Nathan Heaton, Brown Summit, NC (US)

(73) Assignee: Berkshire Holding Corporation, Whitsett, NC (US)

(21) Appl. No.: 18/008,057

(22) PCT Filed: Dec. 30, 2020
(86) PCT No.: PCT/US2020/067413

§ 371 Date: Dec. 2, 2022

Related U.S. Application Data
(60) Provisional application No. 63/034,716, filed on Jun. 4, 2020.

(51) Int. Cl. A61L 2/18; B08B 1/00

(52) U.S. Cl. CPC A61L 2/18; B08B 1/006; A61L 2202/17; A61L 2202/181

(57) ABSTRACT

A cleaning kit. The cleaning kit including a first liquid-tight container including a solute and a second liquid-tight container including a solvent adapted to be received within the first container to be ruptured by the application of pressure to the container by a user. The second container includes a sealable liquid-tight pouch; and a frangible seal along at least a portion of an outer edge of the pouch, wherein the frangible seal is broken by the application of pressure to the second container by a user greater than about 30 inches of water when tested according to ASTM Test F1140-07 (2007), wherein the second container is adapted to be openable while it is located in the first container to release the solvent from the second container into the first container, whereby the solute and the solvent are combined to form a cleaning liquid.

### BACKGROUND OF THE INVENTIONS

### (1) Field

The present inventions relate generally to cleaning devices and methods, and more particularly to such devices and methods used in cleaning “clean rooms”, semiconductor fabrication plants, pharmaceutical manufacturing facilities, and other cleaning applications and environments where extreme cleanliness is maintained.

### (2) Related Art

The requirements for maintaining cleanliness in semiconductor fabrication clean rooms, pharmaceutical manufacturing clean rooms and similar facilities are stringent. In semiconductor fabrication clean rooms, surfaces frequently must be wiped with an exceptionally clean wiper and cleaning solution in order to prevent contamination. The contamination which is to be controlled often is called “micro-contamination” because it consists of small physical contaminants, such as particulate matter of a size between that of bacteria and viruses, and chemical contaminants in very low concentrations, typically measured in parts per million or parts per billion. The contaminants usually are of three types; viable and non-viable particles, ions and non-volatile residues. These contaminants are deposited in the clean room and on products and surfaces to be cleaned by a host of sources including raw materials, previous processing steps, manufacturing equipment, and humans in the clean room. The wipers must be very clean to minimize the introduction of additional contaminants. Loose particles 100 micrometers and smaller in size are an anathema to obtaining high production yields and reliable semiconductor devices. Therefore, cleaning applicators and cleaning materials used in such clean rooms should emit as small a number of particles as possible. Similarly, contaminants interfere with the exacting processes of pharmaceutical and semiconductor manufacturing, and their quantities are to be minimized.

In the past, such requirements have been met by the provision of specially fabricated cloths designed to release very few loose particles, while maintaining structural integrity when wetted with cleaning solution and being used to wipe the surfaces to be cleaned. Typically, such wipers have been packaged in sealed containers such as flexible plastic bags, which can be opened, a wiper withdrawn and the bag resealed. Then, the cleaning solution, kept in a separate container, is applied to the wiper and the wiper is used.

Substantial quantities of chemical solvents used as cleaning fluids must be stored and applied to the wipers. This can result in accidents or working conditions deleterious to the health of the workers. Also, when workers apply cleaning fluids from separate containers to dry wiper cloths, the amount of cleaning fluid used often is either too little or too much, which can result in ineffective cleaning, or oversaturation and waste.

In response to these issues, pre-saturated wipers have been developed and commercialized. The wipers are pre-wetted with varying amounts of cleaning solutions and packaged in a re-sealable plastic bag until needed. Then, the bag is opened, the wiper or wipers withdrawn, and the bag is re-closed. This is repeated until the bag is empty. Unfortunately, bags of pre-saturated wipers often may be stored for prolonged periods, such as months or even years before use. During that time, the cleaning liquid and the wiper material are in intimate contact with one another. It is believed that this often leads to a degradation of the fibers of the wiper material such that the quantity of contaminants released by the wiper when used is substantially higher than when the wipers are first packaged. It is believed that the bonds holding the particles to the fibers are loosened, and the releasable ions and extractables are increased by prolonged contact between the cleaning solutions and the wipers.

In addition, where adhesives are used to fasten various parts of plastic packaging together, such as in the re-closable seal of some wiper packages, the adhesive may deteriorate due to prolonged contact with the cleaning liquid.

Another issue of cleaning pharmaceutical manufacturing and other medical facilities with pre-saturated wipers lies in maintaining the effectiveness of the biocide. More specifically, the effectiveness of the biocide may deteriorate due to its reactivity in the solution and/or prolonged contact with the wiper material and therefore cannot provide a meaningful long term shelf life as a commercial product.

In order to overcome the various shortcomings of pre-saturated wipers, others have attempted to include both the unsaturated wipers and cleaning fluid in a single system, but with the wipers and cleaning fluid kept separate until immediately before use. One such system is set forth in U.S. Pat. No. 6,602,381 issued to Paley et al., the disclosure of which is hereby incorporated by reference in its entirety.

Challenges with these “point-of-use saturation” systems include leakage, bulky designs that are expensive to manufacture and ship, and difficulty in saturating the wipers with cleaning fluid prior to use. In particular, premature rupturing of the cleaning fluid compartment during shipping and handling is a serious problem, with known systems exhibiting a 40% failure rate when tested according to ASTM Test Protocol D5276-98 (2009) for Drop Test of Loaded Containers by Free Fall.

One way to protect against rupture during shipping is to employ bubble wrap or other special packaging to provide cushioning. Another way to protect against rupture during shipping is to minimize the quantity of packs in a shipped case in order to reduce weight and thus lessen the pressure on the contents when jostled during shipping. Unfortunately, cushioned packaging and/or shipping smaller quantities adds to shipping and materials expenses.

Another way to protect against rupture during shipping is to employ durable cleaning fluid packaging. Unfortunately, however, this durability likewise makes it difficult for the user to rupture the package in use. If the cleaning fluid packaging isn't effectively ruptured, then the wipers may not be properly saturated. However, the above inventions do not provide solutions to systems where a biocide solution lacks sufficient stability and therefore requires separation of the biocide as a solute from the bulk aqueous solvent and other ingredients.

Thus, there remains a need for a new and improved cleaning kit system including a first liquid-tight container including a solute that receives (1) a second liquid-tight container including a solvent and (2) one or more applicators, the second liquid-tight container adapted to reduce accidental rupture during shipping and handling while, at the same time, being adapted to be easily ruptured by the application of pressure by a user to the first liquid-tight container, whereby the solute and the solvent are combined to form a cleaning liquid.

### SUMMARY OF THE INVENTIONS

The present inventions are directed to a cleaning kit, the kit including a first liquid-tight container including a solute and a second liquid-tight container including a solvent adapted to be received within the first container and to be ruptured by the application of pressure to the container by a user. The second container includes (i) a sealable liquid-tight pouch; and (ii) a frangible seal along at least a portion of an outer edge of the pouch, wherein the frangible seal is broken by the application of pressure to the second container by a user greater than about 30 inches of water when tested according to ASTM Test F1140-07 (2007), wherein the second container is adapted to be openable while it is located in the first container to release the solvent from the second container into the first container, whereby the solute and the solvent are combined to form a cleaning liquid. In addition, there may be at least one absorbent applicator in the first container and adjacent to the second container in the first container whereby the cleaning liquid is applied to the applicator in the first container.

The cleaning kit may further include a baffle extending at least partially across the pouch adapted to reduce accidental rupture caused by handling during shipping, wherein the baffle is selected from the group consisting of a partial weld line, a plurality of spaced apart dashed weld lines, a plurality of scattered weld lines, a single weld dot, a plurality of spaced apart weld dots, a single knurled seam, a plurality of knurled seams, a plurality of intersecting weld lines, and combinations thereof.

In one embodiment, the welds are formed by joining together opposite faces of the pouch by heat sealing, RF welding, ultrasonic welding, adhesives and combinations thereof.

In one embodiment, the baffle is discontinuous in use. The baffle may partially extend across a portion of the pouch. Also, the baffle may extend fully across the pouch and the baffle is adapted to be ruptured by the application of pressure by a user to the cleaning kit.

The pouch may be a pouch formed by sealing at least pair of opposing edges of sheets of material and at least a portion of the sealed edge of the pouch is a frangible seal. Also, the top edge of the pouch may include a frangible seal that is created after the container is filled with a liquid. In one embodiment, the pouch is a fold pouch and at least a portion of the sealed edge of the fold pouch is a frangible seal.

In one embodiment, the baffle is adapted to provide an effective volume after filling with a liquid of between about 30% and about 80% and the strength of the frangible seal is between about 30 inches of water and about 80 inches of water when tested according to ASTM test F1140-07 (2007). The baffle may also be adapted to provide an effective volume after filling with a liquid of between about 40% and about 70% and the strength of the frangible seal is between about 40 inches of water and about 70 inches of water when tested according to ASTM test F1140-07 (2007). The baffle may also be adapted to provide an effective volume after filling with a liquid of between about 30% and about 65% and the strength of the frangible seal is between about 45 inches of water and about 65 inches of water when tested according to ASTM test F1140-07 (2007). The baffle may also be adapted to provide an effective volume after filling with a liquid of about 60% and the strength of the frangible seal is about 55 inches of water when tested according to ASTM test F1140-07 (2007).

In one embodiment, the solute is selected from the group consisting of stable inorganic or organic chlorine-containing substances that may react and release chlorine disinfectants upon dissolution in the solvent and combinations thereof. The solute may be selected from the group consisting of Ca(ClO)₂, NaClO₂, sodium dichloroisocyanuric acid and its derivatives, etc. and combinations thereof. In on embodiment, the solute is selected from the group consisting of sodium dichloroisocyanurate (NaDCC) and combinations thereof.

The solute may further include an inert matrix. In one embodiment, the inert matrix is selected from the group consisting of pH adjustors (acids and alkalis), processing aids (salts and minerals), polymers (e.g. polyvinyl alcohols), preservatives, surfactants, and additional biocides with residual efficacy (e.g. quaternary amines, copper or silver salts) and combinations thereof.

In one embodiment, the solvent is selected from the group consisting of water and aqueous solutions of other substances that may react and release chlorine disinfectants upon dissolving the solute and combinations thereof.

The solvent may further include a supplemental cleaning agent. In one embodiment, the supplemental cleaning agent is selected from the group consisting of acids, alkalis, buffers, surfactants, salts, oxidation agents, reducing agents, quenchers, solvents, etc. and combinations thereof.

In one embodiment, the particle size of the solute is between about 4000 and 75 microns. The particle size of the solute may be between about 2000 and 250 microns. In one embodiment, the particle size of the solute is between about 400 and 250 microns.

In one embodiment, the applicator is selected from the group consisting of wipers, mops, swabs, gloves, mitts, pads and combinations thereof. In one embodiment, the applicator is a wiper for use in clean rooms requiring the use of wipers producing very small quantities of contaminants in use. The applicator may include a plurality of the wipers forming a stack. Also, the applicator may be made from materials selected from the group consisting of knitted fabrics, woven fabrics, non-woven fabrics, foams, meshes, sponges and combinations thereof.

In one embodiment, the first liquid-tight container is a pouch having an opening for receiving the second liquid-tight container. The pouch may be a fold pouch formed from a single sheet of material having the edges joined together to form sides and the opening. In one embodiment, the pouch is a 3-side sealed pouch formed from two sheets of material having the edges joined together to form sides and the opening.

The opening of the pouch may be sealable across the opening after receiving the second liquid-tight container. The pouch may also be re-sealable after opening the first container during use. In one embodiment, the pouch is a re-sealable peel and seal pouch.

The pouch may further include a flap normally covering the opening and a pressure sensitive adhesive adapted to re-seal the first container after opening. In one embodiment, the pouch includes a re-sealable slider zipper adapted to re-seal the first container after opening. In one embodiment, the pouch includes a re-sealable pressure zipper adapted to re-seal the first container after opening.

The pouch may be formed from a plastic film. In one embodiment, the plastic film is a single layer plastic film. In one embodiment, the plastic film is a multilayer, composite plastic film. The plastic film may be a thermoplastic film.

In one embodiment, the second liquid-tight container includes a solvent adapted to be received within the first container is positioned between the at least one absorbent applicator in the first container and the solute.

In another embodiment, the second liquid-tight container includes a solvent adapted to be received within the first container is positioned between the at least one absorbent applicator in the first container and the solute and wherein the at least one absorbent applicator in the first container further includes a portion of the solute distributed within the at least one absorbent applicator in the first container.

In another embodiment, the second liquid-tight container includes a solvent adapted to be received within the first container is positioned adjacent to the at least one absorbent applicator in the first container and at least a portion of the solute is distributed onto the inner surface of the first container.

In still another embodiment, the second liquid-tight container includes a solvent adapted to be received within the first container is positioned adjacent to the at least one absorbent applicator in the first container and at least a portion of the solute is distributed onto the inner surface of the at least one absorbent applicator.

Accordingly, one aspect of the present inventions is to provide a cleaning kit, the kit including (a) a first liquid-tight container including a solute; and (b) a second liquid-tight container including a solvent adapted to be received within the first container, wherein the second container is adapted to be openable while it is located in the first container to release the solvent from the second container into the first container, whereby the solute and the solvent are combined to form a cleaning liquid.

Another aspect of the present inventions is to provide a cleaning kit, the kit including (a) a first liquid-tight container including a solute; (b) a second liquid-tight container including a solvent adapted to be received within the first container and to be ruptured by the application of pressure to the container by a user, the container including (i) a sealable liquid-tight pouch; and (ii) a frangible seal along at least a portion of an outer edge of the pouch, wherein the frangible seal is broken by the application of pressure to the second container by a user greater than about 30 inches of water when tested according to ASTM Test F1140-07 (2007) wherein the second container is adapted to be openable while it is located in the first container to release the solvent from the second container into the first container, whereby the solute and the solvent are combined to form a cleaning liquid; and (c) at least one absorbent applicator in the first container and adjacent to the second container in the first container whereby the cleaning liquid is applied to the applicator in the first container.

Still another aspect of the present inventions is to provide a cleaning kit, the kit including (a) a first liquid-tight container including a solute; (b) a second liquid-tight container including a solvent adapted to be received within the first container and to be ruptured by the application of pressure to the container by a user, the container including (i) a sealable liquid-tight pouch; and (ii) a frangible seal along at least a portion of an outer edge of the pouch, wherein the frangible seal is broken by the application of pressure to the second container by a user greater than about 30 inches of water when tested according to ASTM Test F1140-07 (2007) wherein the second container is adapted to be openable while it is located in the first container to release the solvent from the second container into the first container, whereby the solute and the solvent are combined to form a cleaning liquid; and (c) at least one absorbent applicator in the first container and adjacent to the second container in the first container whereby the cleaning liquid is applied to the applicator in the first container.

These and other aspects of the present inventions will become apparent to those skilled in the art after a reading of the following description of embodiments when considered with the drawings.

### BRIEF DESCRIPTION OF THE DRAWINGS

FIG. 1 is a perspective view of an inner container having a single baffle constructed according to the present inventions;

FIG. 2 is a top view of a cleaning kit showing internal components in phantom;

FIG. 3 is a top view of a cleaning kit with an inner bag having a plurality of baffles;

FIG. 4 is a top view of a cleaning kit with swabs;

FIG. 5 depicts a method according to the present inventions including:

FIG. 5A showing a cleaning kit;

FIG. 5B depicting a user applying pressure to an outer container;

FIG. 5C showing cleaning liquid escaping from a broken frangible seam;

FIG. 5D depicting a user opening an outer container;

FIG. 5E showing a user removing a single wiper; and

FIG. 5F showing a user wiping a surface with a wiper;

FIG. 6 is a contour plot of a response surface graphically illustrating overall performance of ease of opening and shipping and handling durability on a scale of 1-5 as a function of the effective volume of the liquid inner contain and the strength of the frangible sea;

FIG. 7 is a cross-sectional view of one embodiment of a cleaning kit with dry wipers and dry bleach source separated from each other by the pouch of activation liquid;

FIG. 8 is a cross-sectional view of another embodiment of a cleaning kit leveraging pre-formed solid bleach components placed across the package and in between individual wipers;

FIG. 9 is a cross-sectional view of another embodiment of a cleaning kit leveraging polymer films coated with solid bleach particles as the outer packaging;

FIG. 10 is a cross-sectional view of another embodiment of a cleaning kit leveraging wipers that are pre-coated with solid-bleach particles or wipers that are made from yarns or fibers that are pre-coated with solid-bleach; and

FIG. 11 is a contour plot of a response surface graphically illustrating overall consistency of bleach level in a wiper pack on a scale of 1-5 as a function of the particle size of the solute and the mixing time of the solute with the solvent combining to form a cleaning liquid before the cleaning liquid wets the wiper pack.

### DESCRIPTION OF THE EMBODIMENTS

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

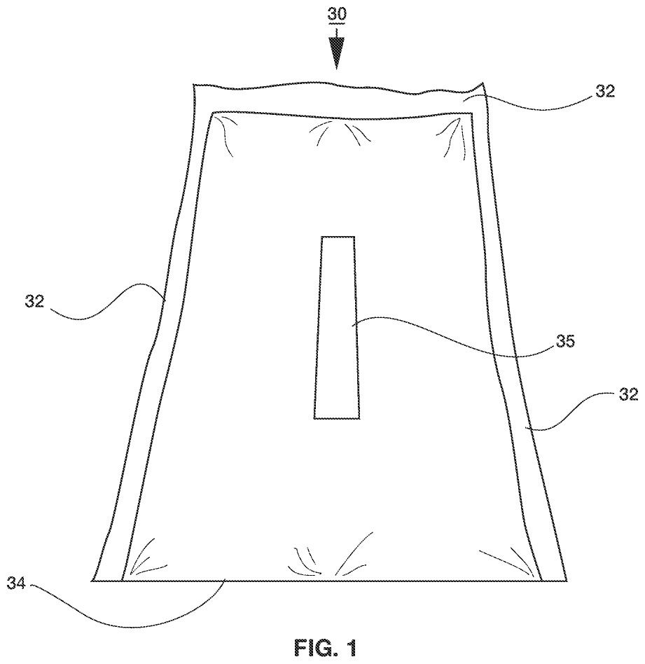

Referring now to the drawings in general and FIG. 1 in particular, it will be understood that the illustrations are for the purpose of describing a preferred embodiment of the inventions and are not intended to limit the inventions thereto. As best seen in FIG. 1, an inner liquid tight, second container 30 for the solvent for the cleaning kit system, generally designated 10 in subsequent figures, is shown constructed according to the present inventions.

Container 30 is preferably a four-sided pouch having three frangible seams 32 and one folded edge 34, although other configurations are within the scope of this invention. As used herein, “frangible seam”, “frangible seal” and the like shall refer to a seam that purposefully comes apart when a pre-determined amount of pressure is applied, rather than deforming elastically and retaining its cohesion as a single object. When force is applied to second container 30, for example through a user exerting pressure on first container 30, at least one frangible seam 32 is compromised and solvent is evacuated from container through broken frangible seam. It should be understood that it is not necessary that all frangible seams, or even one frangible seam in their entirety, be compromised. Rather, a portion of one seam compromised may be adequate to allow evacuation of solvent from second container.

Second container 30 is preferably formed of a piece of plastic film that is folded over and frangibly sealed along three edges to form a pouch with a frangible seal along at least a portion of an outer edge of the pouch. This film is preferably a liquid impermeable thermoplastic. Examples of suitable films include polyester, polypropylene, polyethylene, polyamide, TTFE, or laminates or coforms thereof.

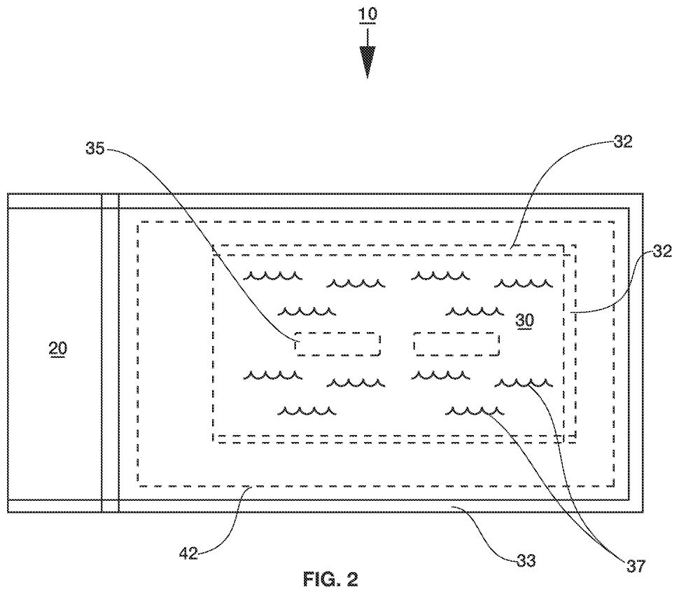

Second container 30 envelops a volume of solvent 37 within, as shown in FIG. 2. As used herein, “solvent” refers to water or aqueous solutions of other substances and their combinations that would react with solid bleach to release the biocidal components, as well as ingredients that would deliver other cleaning benefits: acids, alkalis, buffers, surfactants, salts, oxidation agents, reducing agents, quenchers, solvents, etc.

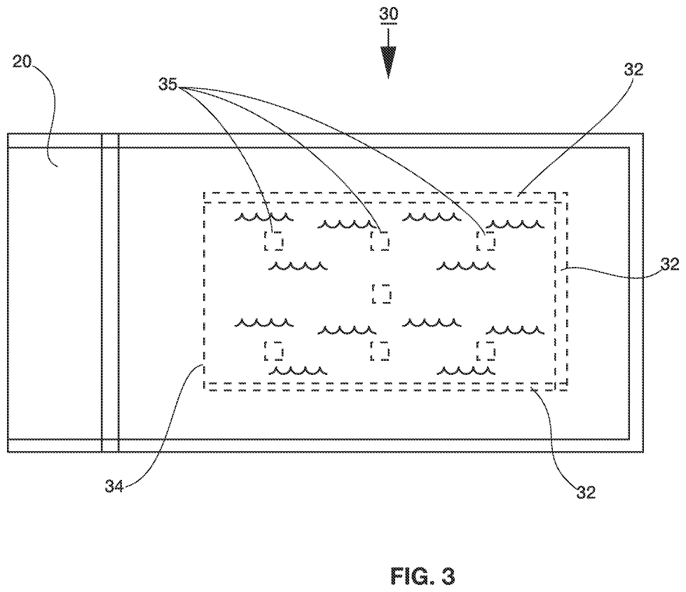

Second container 30 contains at least one baffle 35, but two baffles, or a plurality of baffles, as shown in FIGS. 2 and 3 respectively, are also within the scope of the present inventions. Baffles 35 are preferably formed by welding the two sides of film together, optionally with the resulting baffle seal being slightly weaker than the frangible seam 32. Said another way, when pressure is applied to second container 30, the baffle weld will give way before frangible seams give way. Thus, the “rupturing” event of the second container can typically occur in two parts: the baffle weld or portion thereof will come apart, then the frangible seal will give way and allow the liquid to escape. Baffles may be oriented in a variety of ways including a partial weld line, a plurality of spaced apart dashed weld lines, weld lines scattered and/or at angles to each other, a plurality of spaced apart weld dots, knurled seams, a plurality of intersecting weld lines, a plurality of spaced weld lines, one or more curved weld lines, and combinations thereof. Baffles may be formed by joining together opposite faces of the inner container by means such as heat sealing, RE welding, ultrasonic welding, adhesive, and combinations thereof.

It is desirable that the baffle is “discontinuous in use”. As use herein, “discontinuous in use” shall mean that the baffle doesn't significantly impede the movement of flow within the second container when the frangible seam of the second container is compromised. Said another way, if in use a user applies pressure to the second container so the baffles and frangible seams ‘give way’, the baffles that have “given way” can't significantly prevent the solvent from draining out of the second container through broken frangible seam. Likewise if baffles are designed remain intact during rupturing of frangible seam(s), the baffles can't be oriented in a manner that would prevent evacuation of virtually all of the solvent from the second container when the frangible seams have given way. A discontinuous baffle can extend only partially across a portion of the inner container or extend fully across the inner container,

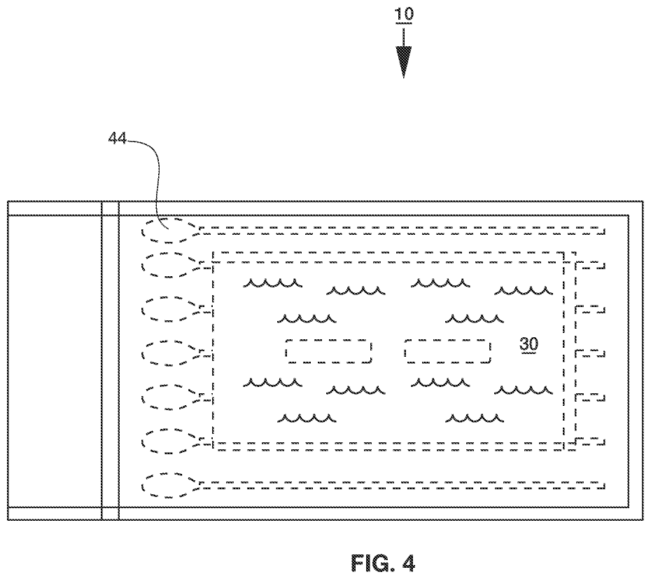

As shown in FIG. 2, cleaning kit 10 includes second container 30, and at least one wiper 42, within first container 20. While wiper 42 is generally used herein for conciseness, it should be understood that the present inventions pertain to a cleaning kit having a variety of cleaning applicators 40 including mops 43, swabs 44 (FIG. 4), gloves 45, mitts 46 and pads 47, in addition to wipers 42, Applicators can be constructed of a variety of materials including knitted fabrics, woven fabrics, non-woven-fabrics, foams, meshes, sponges and combinations thereof. Wipers may be very low quantity contaminant wipers approved for use as clean room wipers in accordance with IEST-RP.CC004.3. One cleaning kit may contain more than one type of cleaning applicators, Cleaning applicators 40 may be provided singly or as a plurality in each first container. Where a plurality of cleaning applicators are provided, a specific configuration may be preferred, for example a stack of wipes. Applicators are preferably absorbent with respect to the cleaning liquid formed by combining the solute and the solvent,

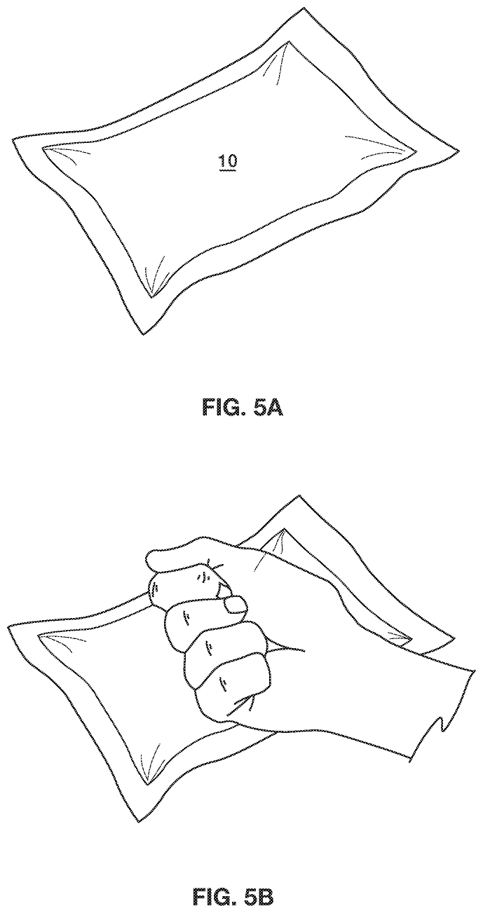

First container 20 is preferably constructed of a liquid impermeable plastic film such as laminated PET/LLDPE film, or PET, PA, PP, CoPET variations, that is preferably sealed on peripheral edges with standard (non-frangible) seam 33. Preferably, the plastic film is a thermoplastic. The plastic film may be a multilayer composite plastic film. In one embodiment, first container 20 is formed from a piece of plastic film having its outer edges joined together and including an opening to form a pouch through which second container 30 is inserted. Alternatively, first container 20 can be formed of two sheets of material having joined edges. First container 20 preferably includes access opening 22 (FIG. 5D) through which cleaning applicators 40 may be removed. FIG. 5 will be discussed in more detail infra.

Opening 22 is preferably closed using seal 23. Seal 23 can be a variety of configurations including re-sealable “peel and seal” flaps, pressure sensitive adhesives, slider zippers, and combinations thereof. Opening 22 can preferably be opened and reclosed using seal 23 multiple times. Second container 30 is enclosed within first container 20.

The effective volume of solvent within the second container is a crucial aspect of the present inventions. If the effective volume is too low, then it is difficult for the user to rupture the inner container for use. Too low volumes will also result in more time required to achieve uniform saturation and may limit the number of wipers in the pack for a desired saturation level. If the effective volume is too high, then the second container is likely to rupture before desired, for example during shipping. As used herein “effective volume” shall refer to the maximum volume of fluid in a baffled container versus the maximum volume of fluid in that container without a baffle, expressed as a percentage. By way of example, if a 1 liter container with baffles has a maximum volume of 0.7 liters, the effective volume would be 70%. This is calculated as follows:

(V_{Baffle}/V_{Unbaffled})×100=Effective Volume

Likewise, the strength of frangible seam 32 is an aspect of the present inventions. If the frangible seam is too strong then it is difficult for the user to rupture the inner container for use, but if it is too weak then the second container is likely to prematurely rupture.

Extensive experimentation was necessary to achieve both ease of use and shipping and handling durability. “Ease of use” pertains to how easy it is for a user, for example a cleaning technician in a clean room, to rupture the second container for purposes of allowing solvent to come in contact with solute and cleaning applicator. “Shipping durability” refers to how resilient the second container is during the shipping and handling process, as defined by ASTM Test Protocol D5276-98 (2009) for Drop Test of Loaded Containers by Free Fall. Ease of use and shipping durability are inherently contradictory qualities.

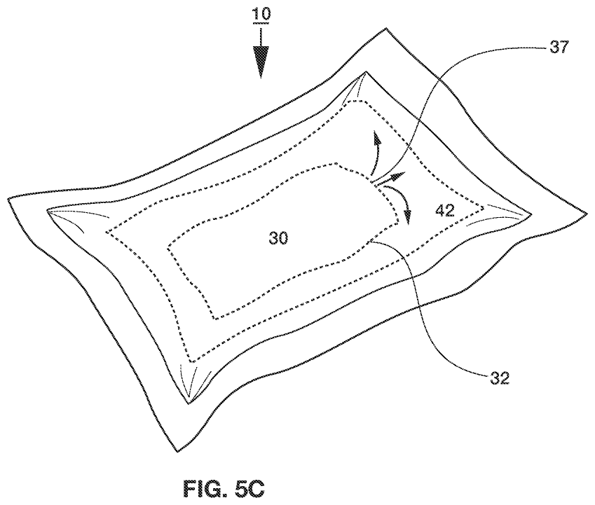

As best seen in FIG. 6, there is shown a contour plot of a response surface graphically illustrating overall performance of ease of opening and shipping and handling durability on a scale of 1-5 as a function of the effective volume of the liquid inner contain and the strength of the frangible seal. FIG. 6 quantifies overall performance on a 1-5 scale, with 5 being the best.

It should be understood that the addition of baffles is a critical element of the invention insofar as it permits both ease of use and shipping durability. Previous technologies optimized one at the expense of the other.

It is desirable that the inner container of the present inventions has an effective volume of approximately 30%-80%, and a frangible seam strength of approximately 30-80 inches of water. It is more desirable that the inner container of the present inventions has an effective volume of approximately 40%-70%, and a frangible seam strength of approximately 40-70 inches of water. It is even more desirable that the inner container of the present inventions has an effective volume of approximately 45%-65%, and a frangible seam strength of approximately 45-65 inches of water. It is most desirable that the inner container of the present inventions has an effective volume of approximately 60%, and a frangible seam strength of approximately 55 inches of water. As used herein, frangible seal strength is determined by ASTM Protocol F1140-07 (2007).

As demonstrated in the following prophetic tables, the percent of pouch volume capacity is also an important factor in performance. In these tables a rating of 1 is unacceptable; 2 is a 10% failure rate; 3 is a 1% failure rate; 4 is a <0.023% failure rate; and 5 is excellent performance. As used herein “percent of pouch volume capacity” shall refer to the percent capacity that a pouch is filled. For example, if a baffled or unbaffled pouch has a maximum capacity of 1 liter, but is filled with 0.8 liters, the percent of pouch volume capacity would be 80%. This is calculated as followed:

(V_{Actual}/V_{Capacity})×100=Percent of Pouch Volume Capacity

Referring back again to FIG. 5, FIG. 5A depicts the cleaning kit having a second container 30 enclosing a volume of solvent 37 and solute 38 and at least one cleaning applicator wiper 42. FIG. 5B depicts a user applying force to the cleaning kit for purposes of rupturing second container 30 to release solvent within whereby the solute and the solvent are combined to form a cleaning liquid. It should be understood that the application of force can be achieved by a variety of methods including folding, striking, twisting, and pushing the cleaning kit with hands, other objects, or a combination. Also, it should be understood that pressure is exerted on first container 20, and that force carries through to second container 30. The user doesn't open first container 20 to access and apply pressure to second container 30. FIG. 5C depicts solvent 37 escaping frangible seam 32 of second container 30, whereby the solute and the solvent are combined to form cleaning liquid and come in contact with wiper 42 within first container 20. FIG. 5D depicts a user peeling back seal 23 to reveal access opening 22. FIG. 5E depicts a user removing a wiper 42. FIG. 5F depicts a user wiping a surface with wiper 42.

In one embodiment of the present inventions, the solute is a solid bleach source and the solvent is water. The present inventions produce cleaning and disinfecting wipers by leveraging a class of solid bleach source, sodium dichloroisocyanurate (NaDCC), whose biocidal benefits are activated upon dissolution in water. The subsequent bleach solution delivers the desired biocidal benefits, but is only stable for a few days, therefore requires dissolution and activation by water prior to use. The inventions include various ways to incorporate the bleach source, activation solutions, and cleaning wipers in one package, although separated, and mechanisms to effectively mix, activate, and dose the wipers with biocidal solutions.

NaDCC is traditionally manufactured in the forms of granules or tablets to ensure shelf-life. The usage involves measuring and dissolving the solids in prescribed amounts of water to produce biocidal solutions. These methods are inconvenient and prone to errors due to multi-step procedures.

NaDCC has also been fashioned in other forms such as sheets, with or without an inert matrix material that would disintegrate upon mixing with water, such as water-soluble polymers, to provide better convenience and flexibility. However, these forms still require measuring water and mixing, therefore provide limited improvement on the first class of technologies.

NaDCC has been directly deposited onto cleaning wipers in forms such as crystals, powers, or pastes. Usage of the products involves wetting the wiper to activate the biocidal ingredients. It is very difficult to achieve quantitative activation such that the biocidal efficacy, which is highly dependent on active ingredient concentration, is guaranteed.

Multi-compartmented configurations separate the components, including the chemicals and oftentimes a cleaning tool such as wipers. All the components can be quantitatively included in the product to ensure correct dosing. These technologies require multiple layers of water-permeable and/or -impermeable materials to create cavities for the various components and breaking certain barriers to accomplish mixing and dosing. Main limitations of these technologies are high costs associated with complexity of configuration, as well as mixing mechanisms that are still not intuitive.

Some prior technologies incorporated a rigid cleaning tool and/or multi-layered wiper materials, to enhance the mechanical strength and usage convenience. Limitations of these forms include bulkiness of the product and high cost to manufacture.

In one embodiment, the invention is composed of 3 key components: wiper substrates that are cut and arranged in a ready-to-dispense fashion; a solid bleach source that is judiciously placed within package to be activated and distributed in a controllable fashion; and a liquid formula capable of activate solid bleach and deliver other key benefits.

NaDCC does have a wide range of dilution ratios. However, end users will follow label instructions for specific biocidal benefits. The relevant range is roughly 1/2000 to 1/100, w/w of solute to solvent. The activity of the resulting cleaning liquid is typically measured by Cl%. The chlorine levels would be higher than that in drinking water but not close to saturation.

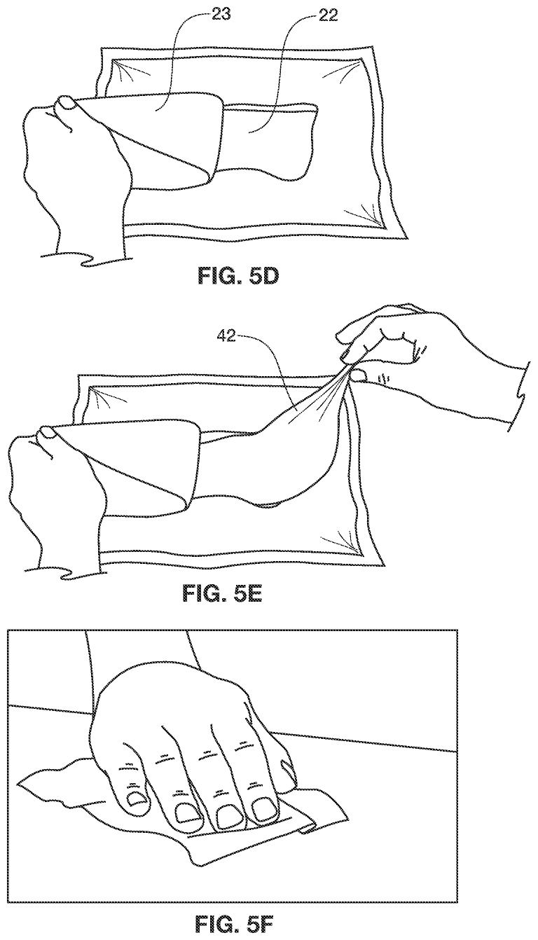

As best seen in FIG. 7, there is shown a cross-sectional view of one embodiment of a cleaning kit with dry wipers and dry bleach source separated from each other by the pouch of activation liquid. Criteria for this design include: 1) wiper substrates with appropriate rigidity and absorption rate, e.g. polypropylene or polyester non-wovens are selected; 2) NaDCC solids are placed at the far end of package and insolated with the activation liquid pouch from the wiper substrates; and 3) the outer package is vacuum-sealed to maintain the relative positions of the key components during transportation. All these design components allow the bleach source sufficient space and time to dissolve before being absorbed by the wipers.

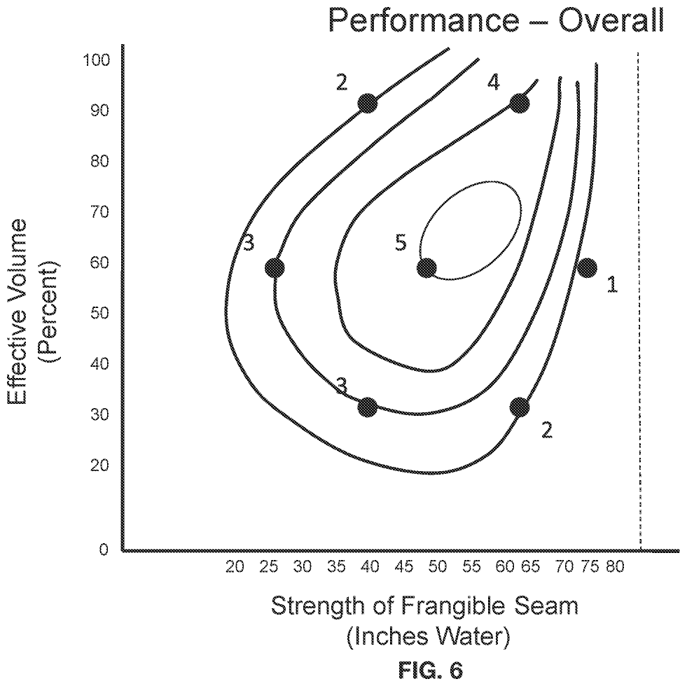

Turning now to FIG. 8, there is shown a cross-sectional view of another embodiment of a cleaning kit leveraging pre-formed solid bleach components, in various shapes such as threads, rods, sheets, nets, etc, whether extruded, compressed, or mixed with a matrix material. These solid bleach components are evenly placed across the package and in between individual wipers. Upon breaking of the activation liquid pouch, these sheets dissolve and evenly distribute bleach solution across the pack. Moreover, if a matrix is used to contain, host, protect or disperse the solid bleach material, more ingredients may be included in the matrix to deliver additional benefits, such as anti-fouling, slow-release, or residual efficacy.

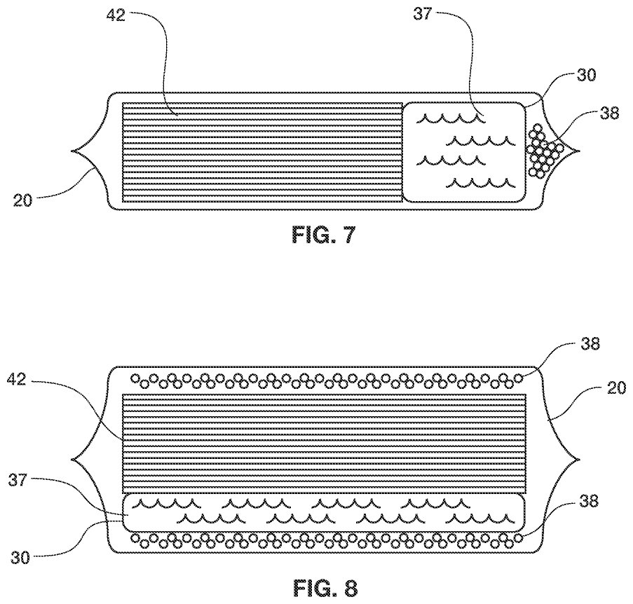

Turning now to FIG. 9, there is shown a cross-sectional view of another embodiment of a cleaning kit leveraging polymer films coated with solid bleach particles as the outer packaging.

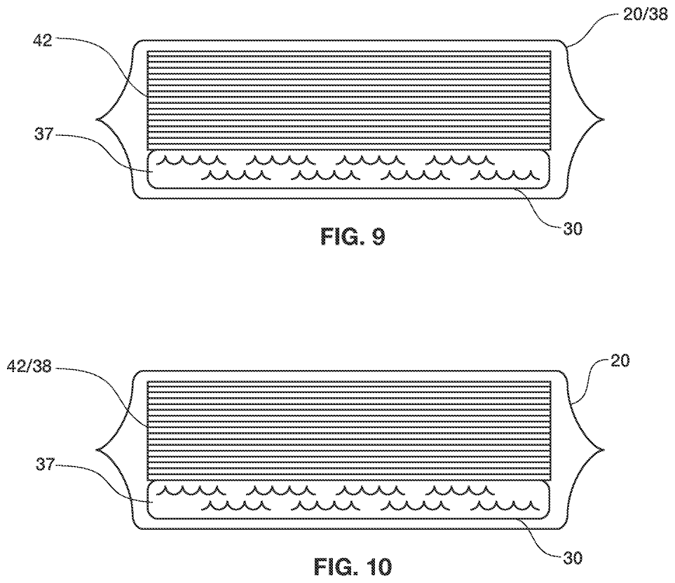

Turning now to FIG. 10, there is shown a cross-sectional view of another embodiment of a cleaning kit leveraging wipers that are pre-coated with solid-bleach particles or wipers that are made from yarns or fibers that are pre-coated with solid-bleach.

In each of the above embodiments, the solid bleach is effectively separated with activation liquid to ensure shelf-life and premature mixing. In addition, the form of the product also allows the solids to sufficiently dissolve before the liquid is absorbed by the wipers. Premature absorption would result in low levels of active ingredients in the wiper.

The form of the product as illustrated in FIGS. 7-10 affects mixing time of the solute with the solvent. This works together with the size of the solute. In one embodiment, the particle size of the solute is between about 4000 and 75 microns. The particle size of the solute may further be between about 2000 and 250 microns for a further improvement. In one embodiment, the particle size of the solute is between about 400 and 250 microns. However, determining whether uniform mixing and distribution of the active ingredients in the wipers will occur for a given product form and particle size of the solute is difficult to predict as discussed below.

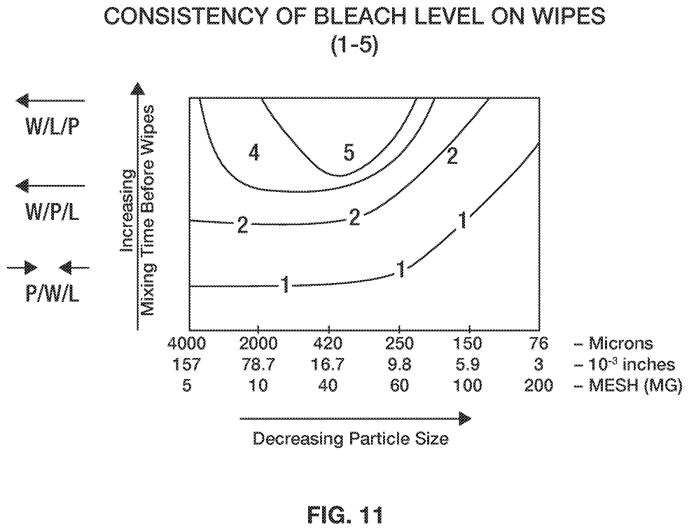

As best seen in FIG. 11, there is shown a contour plot of a response surface graphically illustrating overall consistency of bleach level in a wiper pack on a scale of 1-5 as a function of the particle size of the solute and the mixing time of the solute with the solvent combining to form a cleaning liquid before the cleaning liquid wets the wiper pack. FIG. 11 quantifies overall performance on a 1-5 scale, with 5 being the best.

The X-axis is the particle size of the solute in microns (diameter also shown in mils and mesh size (Market Grade)). The Y-axis is for representative product forms. For example, W/L/P corresponds closely to the specific embodiment shown in FIG. 7 with dry wipers and dry bleach source separated from each other by the pouch of activation liquid which performed very well.

W/P/L corresponds to dry wipers and the pouch of activation liquid separated from each other by the dry bleach source which did not perform as well as W/L/P.

Finally, P/W/L corresponds to dry bleach source and the pouch of activation liquid separated from each other by the dry wipers which performed even more poorly.

In operation, for each embodiment, the pack is first pressured to burst the inner pouch that contains the solvent as shown in FIG. 5. Then the pouch is allowed to sit for minimum 5 minutes such that solid bleach may be dissolved. Prior to use, the pack is squeezed and shaken for a few seconds and the cleaning liquid redistributed, to ensure optimal activation.

The combination of the solid bleach and the disposable wipers deliver biocidal efficacy consistently in convenient, low-cost methods to prepare the above combinations.

Specifically, in comparison to solid bleach tablets or powder, the present inventions allow one step preparation of disinfectant wipers.

Specifically, in comparison to solid bleach sheets or direct deposition onto wipers, the present inventions incorporate pre-measured activation liquids for easy use while, at the same time; introduce novel ways to combine solid bleach with wipers to guarantee consistent dosing.

Specifically, in comparison to multi-compartment, water-activated packages, tools, or devices, the present inventions enable the water-activation in a low-cost, convenient way while maintaining consistent delivery of efficacy.

Certain modifications and improvements will occur to those skilled in the art upon a reading of the foregoing description. By way of example, baffles could be structures other than welds, for example physical barriers such as flaps, objects, gussets, folds, and combinations thereof. Also, the second pouch could be fixed to the first pouch on one or more sides so that the second pouch can't move or shift relative to the first pouch or be accidentally removed from the first pouch. The wiper substrate may be any type of non-woven, knitted, woven, or foam materials that are processible under common cleaning wiper manufacturing conditions, and moderately compatible with the antimicrobial solution. The solid bleach solute may include stable inorganic or organic chlorine-containing substances that may react and release chlorine disinfectants upon dissolution in the activation liquid: Ca(ClO)₂, NaClO₂, sodium dichloroisocyanuric acid and its derivatives, etc. The inert matrix in which the solid bleach source may be mixed may contain pH adjustors (acids and alkalis), processing aids (salts and minerals), polymers (e.g. polyvinyl alcohols), preservatives, surfactants, and additional biocides with residual efficacy (e.g. quaternary amines, copper or silver salts). The solvent of water or aqueous solutions of other substances and their combinations that would react with solid bleach to release the biocidal components, as well as ingredients that would deliver other cleaning benefits: acids, alkalis, buffers, surfactants, salts, oxidation agents, reducing agents, quenchers, solvents, etc. The flexible packaging material for the inner pouch should be compatible with the activation liquid and for the outer pouch should be compatible with the solid bleach and may only need to be moderately compatible with the resulting bleach solutions for the cleaning liquid. While the outer package may be vacuum-sealed to physically help maintain the relative positions of the key components during transportation, other packaging may also be used to maintain the desired relative positions of the wipers; solvent; and solute until use. It should also be understood that “approximately” and other similar terms are +/−10% unless otherwise stated or contrary to common sense. All ranges set forth should be understood to include stated endpoints as well as all increments there between. It should be understood that all such modifications and improvements have been deleted herein for the sake of conciseness and readability but are properly within the scope of the following claims.

We claim:

1. A cleaning kit, said kit comprising:
(a) a first liquid-tight container including a solute; and
(b) a second liquid-tight container including a solvent adapted to be received within said first container; wherein said second container is adapted to be openable while it is located in said first container to release said solvent from said second container into said first container, whereby said solute and said solvent are combined to form a cleaning liquid.

2. The cleaning kit according to claim 1 further including at least one absorbent applicator in said first container.

3. The cleaning kit according to claim 2, wherein said applicator is selected from the group consisting of wipers, mops, swabs, gloves, mitts, pads and combinations thereof.

4. The cleaning kit according to claim 3, wherein said applicator is a wiper for use in clean rooms requiring the use of wipers producing very small quantities of contaminants in use.

5. The cleaning kit according to claim 4, wherein said applicator includes a plurality of said wipers forming a stack.

6. The cleaning kit according to claim 4, wherein said applicator is made from materials selected from the group consisting of knitted fabrics, woven fabrics, non-woven fabrics, foams, meshes, sponges and combinations thereof.

7. The cleaning kit according to claim 1, wherein said first liquid-tight container is a pouch having an opening for receiving said second liquid-tight container.

8. The cleaning kit according to claim 7, wherein said pouch is a fold pouch formed from a single sheet of material having the edges joined together to form sides and said opening.

9. The cleaning kit according to claim 7, wherein said pouch is a 3-side sealed pouch formed from two sheets of material having the edges joined together to form sides and said opening.

10. The cleaning kit according to claim 7, wherein the opening of said pouch is sealable across said opening after receiving said second liquid-tight container.

11. The cleaning kit according to claim 7, wherein said pouch re-sealable after opening said first container during use.

12. The cleaning kit according to claim 11, wherein said pouch is a re-sealable peel and seal pouch.

13. The cleaning kit according to claim 11, wherein said pouch further includes a flap normally covering said opening and a pressure sensitive adhesive adapted to re-seal said first container after opening.

14. The cleaning kit according to claim 11, wherein said pouch includes a re-sealable slider zipper adapted to re-seal said first container after opening.

15. The cleaning kit according to claim 11, wherein said pouch includes a re-sealable pressure zipper adapted to re-seal said first container after opening.

16. The cleaning kit according to claim 7, wherein said pouch is formed from a plastic film.

17. The cleaning kit according to claim 16, wherein said plastic film is a single layer plastic film.

18. The cleaning kit according to claim 16, wherein said plastic film is a multilayer, composite plastic film.

19. The cleaning kit according to claim 16, wherein said plastic film is a thermoplastic film.

20. The cleaning kit according to claim 2, wherein said second liquid-tight container including a solvent adapted to be received within said first container is positioned between said at least one absorbent applicator in said first container and said solute.

21. The cleaning kit according to claim 20, wherein said second liquid-tight container including a solvent adapted to be received within said first container is positioned between said at least one absorbent applicator in said first container and said solute and wherein said at least one absorbent applicator in said first container further includes a portion of said solute distributed within said at least one absorbent applicator in said first container.

22. The cleaning kit according to claim 2, wherein said second liquid-tight container including a solvent adapted to be received within said first container is positioned adjacent to said at least one absorbent applicator in said first container and at least a portion of said solute is distributed onto the inner surface of said first container.

23. The cleaning kit according to claim 2, wherein said second liquid-tight container including a solvent adapted to be received within said first container is positioned adjacent to said at least one absorbent applicator in said first container and at least a portion of said solute is distributed onto the inner surface of said at least one absorbent applicator.

24. A cleaning kit, said kit comprising:
(a) a first liquid-tight container including a solute;
(b) a second liquid-tight container including a solvent adapted to be received within said first container and to be ruptured by the application of pressure to said container by a user, said container including (i) a sealable liquid-tight pouch; and (ii) a frangible seal along at least a portion of an outer edge of said pouch, wherein said frangible seal is broken by the application of pressure to said second container by a user greater than about 30 inches of water when tested according to ASTM Test F1140-07 (2007) wherein said second container is adapted to be openable while it is located in said first container to release said solvent from said second container into said first container, whereby said solute and said solvent are combined to form a cleaning liquid;

25. The cleaning kit according to claim 24, further including a baffle extending at least partially across said pouch adapted to reduce accidental rupture caused by handling during shipping, wherein said baffle is selected from the group consisting of a partial weld line, a plurality of spaced apart dashed weld lines, a plurality of scattered weld lines, a single weld dot, a plurality of spaced apart weld dots, a single knurled seam, a plurality of knurled seams, a plurality of intersecting weld lines, and combinations thereof.

26. The cleaning kit according to claim 25, wherein said welds are formed by joining together opposite faces of said pouch by heat sealing, RF welding, ultrasonic welding, adhesives and combinations thereof.

27. The cleaning kit according to claim 25, wherein said baffle is discontinuous in use.

28. The cleaning kit according to claim 27, wherein said baffle partially extends across a portion of said pouch.

29. The cleaning kit according to claim 27, wherein said baffle extends fully across said pouch and said baffle is adapted to be ruptured by the application of pressure by a user to said cleaning kit.

30. The cleaning kit according to claim 24, wherein said pouch is a pouch formed by sealing at least pair of opposing edges of sheets of material and at least a portion of the sealed edge of said pouch is a frangible seal.

31. The cleaning kit according to claim 30, wherein the top edge of said pouch includes a frangible seal that is created after said container is filled with a liquid.

32. The cleaning kit according to claim 30, wherein said pouch is a fold pouch and at least a portion of the sealed edge of said fold pouch is a frangible seal.

33. The cleaning kit according to claim 24, wherein said baffle is adapted to provide an effective volume after filling with a liquid of between about 30% and about 80% and the strength of said frangible seal is between about 30 inches of water and about 80 inches of water when tested according to ASTM test F1140-07 (2007).

34. The cleaning kit according to claim 33, wherein said baffle is adapted to provide an effective volume after filling with a liquid of between about 40% and about 70% and the strength of said frangible seal is between about 40 inches of water and about 70 inches of water when tested according to ASTM test F1140-07 (2007).

35. The cleaning kit according to claim 34, wherein said baffle is adapted to provide an effective volume after filling with a liquid of between about 30% and about 65% and the strength of said frangible seal is between about 45 inches of water and about 65 inches of water when tested according to ASTM test F1140-07 (2007).

36. The cleaning kit according to claim 35, wherein said baffle is adapted to provide an effective volume after filling with a liquid of about 60% and the strength of said frangible seal is about 55 inches of water when tested according to ASTM test F1140-07 (2007).

37. The cleaning kit according to claim 24, wherein said solute is selected from the group consisting of stable inorganic or organic chlorine-containing substances that may react and release chlorine disinfectants upon dissolution in said solvent and combinations thereof.

38. The cleaning kit according to claim 37, wherein said solute is selected from the group consisting of Ca(ClO)₂, NaClO₂, sodium dichloroisocyanuric acid and its derivatives, etc. and combinations thereof.

39. The cleaning kit according to claim 38, wherein said solute is selected from the group consisting of sodium dichloroisocyanurate (NaDCC) and combinations thereof.

40. The cleaning kit according to claim 24, wherein said solute may further include an inert matrix.

41. The cleaning kit according to claim 40, wherein said inert matrix is selected from the group consisting of pH adjustors (acids and alkalis), processing aids (salts and minerals), polymers (e.g. polyvinyl alcohols), preservatives, surfactants, and additional biocides with residual efficacy (e.g. quaternary amines, copper or silver salts) and combinations thereof.

42. The cleaning kit according to claim 24, wherein said solvent is selected from the group consisting of water and aqueous solutions of other substances that may react and release chlorine disinfectants upon dissolving said solute and combinations thereof.

43. The cleaning kit according to claim 42, wherein said solvent may further include a supplemental cleaning agent.

44. The cleaning kit according to claim 43, wherein said supplemental cleaning agent is selected from the group consisting of acids, alkalis, buffers, surfactants, salts, oxidation agents, reducing agents, quenchers, solvents, etc. and combinations thereof.

45. The cleaning kit according to claim 24, wherein the particle size of said solute is between about 4000 and 75 microns.

46. The cleaning kit according to claim 45, wherein the particle size of said solute is between about 2000 and 250 microns.

47. The cleaning kit according to claim 46, wherein the particle size of said solute is between about 400 and 250 microns.

48. A cleaning kit, said kit comprising:
(a) a first liquid-tight container including a solute;
(b) a second liquid-tight container including a solvent adapted to be received within said first container and to be ruptured by the application of pressure to said container by a user, said container including (i) a sealable liquid-tight pouch; and (ii) a frangible seal along at least a portion of an outer edge of said pouch, wherein said frangible seal is broken by the application of pressure to said second container by a user greater than about 30 inches of water when tested according to ASTM Test F1140-07 (2007) wherein said second container is adapted to be openable while it is located in said first container to release said solvent from said second container into said first container, whereby said solute and said solvent are combined to form a cleaning liquid; and
(c) at least one absorbent applicator in said first container and adjacent to said second container in said first container whereby the cleaning liquid is applied to the applicator in said first container.
